(19) United States
(12) Patent Application Publication
(10) Pub. No.: US 2026/0020573 A1
(43) Pub. Date: Jan. 22, 2026

(54) USE OF AMINO ACID PEPTIDE SOLUTION PRODUCED BY DECOMPOSITION OF FISH SCALE USING BACILLUS VELEZENSIS FOR HEALTHY AND STRONG GROWTH OF GRAMINEAE SEEDLING AND GROWTH PROMOTION OF GRAMINEAE

(71) Applicant: CH BIOTECH R&D CO., LTD., NANTOU COUNTY (TW)

(72) Inventors: HUI-LIANG WANG, NANTOU COUNTY (TW); YA-FANG HONG, NANTOU COUNTY (TW); CHIAO-FANG HSU, NANTOU COUNTY (TW)

(21) Appl. No.: 18/776,795

(22) Filed: Jul. 18, 2024

(51) Int. Cl. A01N 63/50; A01N 63/10; A01N 63/22; A01P 21/00; C12N 1/20; C12P 13/04; C12P 21/06; C12R 1/07

(52) U.S. Cl. CPC A01N 63/50; A01N 63/10; A01N 63/22; A01P 21/00; C12N 1/205; C12P 13/04; C12P 21/06; C12R 2001/07

(57) ABSTRACT

A use of an amino acid peptide solution produced by decomposition of fish scales using Bacillus velezensis for healthy and strong growth of Gramineae seedlings and growth promotion of Gramineae is provided. The Bacillus velezensis is deposited in Leibniz Institute DSMZ-German Collection of Microorganisms and Cell Cultures GmbH with an accession number of DSM 34894. The amino acid peptide solution produced by decomposition of the fish scales using the Bacillus velezensis is composed of peptides and amino acids. While being applied to leaf surfaces of seedlings of Gramineae, the amino acid peptide solution can promote growth and keep healthy and strong growth of the seedlings of the Gramineae effectively and specifically.

### SEQUENCE LISTING INCORPORATED BY REFERENCE

The Sequence Listing written in file “957-3533-SEQUENCE LISTING-JUL18-2024.xml” created on Jul. 15, 2024 with modification date Jul. 18, 2024, file size 3,503 bytes, is hereby incorporated by reference in its entirety for all purposes.

### BACKGROUND OF THE INVENTION

### Field of the Invention

The present invention relates to a use of an amino acid peptide solution, especially to a use of an amino acid peptide solution produced by decomposition of fish scales using Bacillus velezensis. The amino-acid peptide solution is applied to Gramineae to promote seedling growth of the Gramineae and make seedlings of the Gramineae grow healthy and strong. Thus above-ground biomass of the Gramineae is further increased.

### Description of Related Art

Fish scales are removed before cooking and eating fish and the fish scales removed are usually discarded as waste in the past. Recently, there are more and more research related to the fish scales in order to make the best use of the fish scales and reduce the waste.

The fish scales contain about 50% collagen, most of which is produced into collagen peptides by enzymatic hydrolysis, and the collagen peptides are broadly applied to skincare products and health supplements. The fish scales are rarely used as fertilizers for promotion of germination or growth of crops.

As to other fertilizers related to collagen, refer to Chinese Pat. Pub. No. 109890781A which provides an amino acid fertilizer composition prepared by treating livestock farming by-products (such as cowhide, pigskin, or cow bones) with acid, alkali, or protease to give crude collagen peptides and then hydrolyzing the crude peptides with Bacillus species. Thereby a fertilizer composed of crude collagen peptides and amino acids is obtained. Although the fertilizer composition facilitates growth of lettuces, the use of livestock farming by-products may cause zoonoses such as Bovine spongiform encephalopathy (BSE). Compared with the fertilizer from the livestock by-products, fish-based fertilizers have a lower risk of contamination and zoonoses.

Most of fish-based fertilizers available on the market are produced by composite fish materials, including fish skin, fish bone, fish paste, fish powder, and recovered protein from surimi wash water. During manufacturing process, mixed strains such as yeast, lactic acid bacteria, Bacillus amyloliquefaciens, and Lactobacillus acidophilus are used. Thus, the manufacturing process is more complicated. Symbiosis Agx (American brand) is a fertilizer derived from whole freshwater fish while Aminoterra® is derived from enzymatic hydrolysis of salmon. Compared with products obtained by simple hydrolysis of fish scales, composite fish materials may lead to inconsistent quality of fertilizer products and poor reproducibility of effect due to different sources and ratios of raw materials.

Gramineae (Poaceae) plants, e.g., rice, maize, barley, wheat, oat, millet, and sugarcane are not only human food sources but also main sources of animal feed. They can also be applied to various industries, including architecture, pulp and paper, textile, brewing, sugar production, pharmacy, furniture, and weaving. Thus, the Gramineae have a close relationship with our lives. In Gramineae, maize is an annual herbaceous plant and one of the important food crops. According to statistics, the worldwide production of corn in 2021 is about 1.21 billion metric tons, followed by wheat 771 million metric tons and rice 787 million metric tons. Maize shows the highest production among the food crops worldwide. Healthy plants have a higher yield and/or better quality products. The corn seedlings with high productivity should have thicker and wider leaves. Healthy and strong seedlings are more resistant to biotic and abiotic stresses. Thus, both healthy and strong seedlings and growth promotion of corn seedlings are key factors in higher productivity and/or quality of corns.

Based on the important impact of the Gramineae on our lives and the higher crop yield of the Gramineae resulting from the healthy and strong growth of seedlings of the Gramineae, there is room for improvement and there is a need to provide a novel way to allow the seedlings of the Gramineae to grow healthy and strong and promote the growth of the Gramineae.

### SUMMARY OF THE INVENTION

Therefore, it is a primary objective of the present invention to provide a use of an amino acid peptide solution produced by decomposition of fish scales using Bacillus velezensis for helping seedlings of Gramineae grow healthy and strong and promoting seedling growth of Gramineae. The amino acid peptide solution, which is produced by decomposition of the fish scales using the Bacillus velezensis and composed of peptides and amino acids is applied to the Gramineae for healthy and strong growth and growth promotion of the seedlings of the Gramineae.

In order to achieve the above objectives, a use of an amino acid peptide solution produced by decomposition of fish scales using Bacillus velezensis for healthy and strong growth and growth promotion of seedlings of Gramineae is provided. The Bacillus velezensis is deposited in Leibniz Institute DSMZ-German Collection of Microorganisms and Cell Cultures GmbH with an accession number of DSM 34894. A bacterial solution containing the Bacillus velezensis is used to degrade fish scale materials and produce the amino acid peptide solution containing peptides and amino acids. Then, the amino acid peptide solution is applied to leaf surfaces of seedlings of Gramineae to keep healthy and strong growth of the seedlings of the Gramineae and promote growth of the seedlings of the Gramineae effectively and specifically.

In some embodiments, a concentration of the peptides and amino acids in the above fish-scale amino acid peptide solution is ranging from 50 ppm to 500 ppm.

In some embodiments, a concentration of the peptides and amino acids in the above fish-scale amino acid peptide solution is 100 ppm, 250 ppm, or 500 ppm.

In some embodiments, a concentration of the peptides and amino acids in the above fish-scale amino acid peptide solution is 100 ppm.

In some embodiments, the Gramineae mentioned above includes rice, oat, wheat, barley, rye, sorghum, millet, sugarcane, maize, Job's tears, lemongrass, bamboo, water bamboo, herbage, tall fescue, bentgrass, and annual meadow grass.

The present invention features on that the amino acid peptide solution produced by decomposing fish scales using Bacillus velezensis is rich in peptides and amino acids. By spraying the amino acid peptide solution on leaf surfaces of seedlings of the Gramineae, not only the seedlings of the Gramineae grow healthier and stronger, but the growth of above-ground biomass of the Gramineae is also promoted. Field management and yield of the Gramineae are further improved.

### BRIEF DESCRIPTION OF THE DRAWINGS

The structure and the technical means adopted by the present invention to achieve the above and other objects can be the best understood by referring to the following detailed description of the preferred embodiments and the accompanying drawings, wherein:

FIG. 1 are photos showing observation of a colony of strain CHB200 and observation of the strain CHB200 with a microscope according to the present invention;

FIG. 2 is a figure showing analysis of contents of amino acids and peptides in decomposition products of fish scales by strain CHB200 according to the present invention;

FIG. 3 are photos showing products obtained at different stages of large-scale fermentation of fish scales by strain CHB200 according to the present invention.

FIG. 4 is a photo showing the growth of corn seedlings in control and treatment groups at day 28 after the corn seedlings were respectively sprayed with water (the control group) and 100 ppm fish-scale amino acid peptide solution twice on leaf surfaces and grown under conditions with sufficient light and water according to the present invention;

FIG. 5 are bar charts showing the number of the leaves (A), a diameter of the stem (B), and a height of seedlings (C) of the corn seedlings respectively sprayed with water (the control group) and 100 ppm fish-scale amino acid peptide solution twice on leaf surfaces; the number of repetitive test samples is 10 (n=10, *p<0.05; **p<0.01; ***p<0.001) according to the present invention;

FIG. 6 are bar charts showing fresh weight (A) and dry weight (B) of the corn seedlings respectively sprayed with water (the control group) and 100 ppm fish-scale amino acid peptide solution twice on leaf surfaces; the number of repetitive test samples is 10 (n=10, *p<0.05; **p<0.01; ***p<0.001), according to the present invention.

### DETAILED DESCRIPTION OF THE PREFERRED EMBODIMENT

A use of an amino acid peptide solution produced by decomposition of fish scales using Bacillus velezensis to promote growth and keep healthy and strong growth of seedlings of the Gramineae is provided. A bacterial solution containing the Bacillus velezensis is mixed with a fish scale material to form a mixed solution. After the fish scale material is decomposed by fermentation of the Bacillus velezensis in the bacterial solution, a fish-scale amino acid peptide solution containing peptides and amino acids is obtained. The fish-scale amino acid peptide solution is applied to leaf surfaces of seedlings of the Gramineae to help the seedlings grow healthy and strong and promote growth of the seedlings of the Gramineae effectively and specifically. The above-ground biomass of the Gramineae is also increased. A concentration of the peptides and amino acids of the fish-scale amino acid peptide solution is ranging from 50 ppm to 500 ppm. The Bacillus velezensis is deposited in Leibniz Institute DSMZ-German Collection of Microorganisms and Cell Cultures GmbH with an accession number of DSM 34894.

16S rRNA of the present Bacillus velezensis strain has 98% similarity with 16S rRNA of type strain (standard strain) of Bacillus velezensis B268 with an accession number of CP053764.1. This standard strain is not disclosed to have fish scale decomposing ability.

The mixed solution is fermented at 30-45° C. for 12-24 hours. The mixed solution includes contains 7-10% (w/v) of the bacterial solution of Bacillus velezensis, 15-40% (w/v) of the fish scale material, 0.12% (w/v) of buffer salts, and the rest percentage of pure water.

After steam sterilization, removal of debris by centrifugation, filtration, and concentrated, a clear and concentrated fish-scale amino acid peptide solution is obtained. Total concentration of peptides and amino acids in the fish-scale amino acid peptide solution is measured by o-Phthaldialdehyde (OPA) test reagent and total concentration of the peptides and amino acids is 30,000 ppm.

In a preferred embodiment, the fish scale material includes fish scales, sodium chloride (NaCl), dipotassium phosphate (K₂HPO₄), and monopotassium phosphate (KH₂PO₄).

In a preferred embodiment, the fish scale material includes fish scales, 0.5 g/L of sodium chloride (NaCl), 0.3 g/L of dipotassium phosphate (K₂HPO₄), and 0.4 g/L of monopotassium phosphate (KH₂PO₄).

The strain CHB200 of Bacillus velezensis is obtained from waste chicken feathers. As to techniques for separating and screening strain CHB200 of Bacillus velezensis, please refer to U.S. patent application Ser. Nos. 18/760,688 and 18/772,775 filed on Jul. 1, 2024, and Jul. 15, 2024, respectively.

Moreover, as to test content of fish scale fermentation ability of the strain CHB200, please refer to U.S. patent application Ser. Nos. 18/760,688 and 18/772,775 filed on Jul. 1, 2024, and Jul. 15, 2024, respectively.

In order to learn functions and features of the present invention more completely and clearly, please refer to the following detailed descriptions and related figures. Yet the following embodiments are not intended to limit the scope of the present invention.

<Isolation and Screening of Bacillus velezensis CHB200>

Collect waste chicken feather. After washing, place 5 g of feathers into a conical flask and add 50 mL of sterile water to get a culture medium. Place the culture medium in a 37° C. incubator and culture overnight with a shaking speed at 200 rpm. Then, remove the feathers from the culture medium and centrifuge the culture medium. Remove supernatant and retain precipitate after centrifugation. Next, dissolve the precipitate with 100 μL sterile water and take and spread 20 μL of re-dissolved solution evenly in Lysogeny broth (LB) solid medium. Incubate the LB solid medium in the 37° C. incubator for 24 hours.

Pick a single colony grown on the LB solid medium, inoculate the colony in 3 ml of LB broth, and culture at 37° C. with a shaking speed at 200 rpm for 24 hours to get a bacterial solution.

Measure absorbance of the bacterial solution at a wavelength of 600 nm (known as OD (optical density) 600). Inoculate the bacterial solution with OD₆₀₀ of 10 therein into 100 mL of M3 fermentation medium containing 33% of fish scale powder and ferment at 37° C. for 48 hours. Then, perform hydrolysis test of fish scales. A concentration of the above bacterial solution with OD₆₀₀ of 10 is about 1×10⁹ CFU/mL and a composition of the M3 fermentation medium is shown in Table 1. At last, measure total concentration of amino acids in fermented solution to screen out a strain with fish scale decomposition ability.

TABLE 1

| | | concentration |
|---|---|---|
| | composition | (g/L) |
| substrate | fish scale | 330 |
| salts and buffer | NaCl | 0.5 |
| | K₂HPO₄ | 0.3 |
| | KH₂PO₄ | 0.4 |

A method of testing total concentration of amino acids is described briefly below.

Dissolve leucine in double-distilled water and prepare amino acid standard solutions with concentrations of 100-2000 ppm by serial dilution. Take 3 μL of amino acid standard solution or test sample (fermented solution), add 87 μL of ninhydrin reagent, and mix evenly to get an intermediate mixture. The ninhydrin reagent includes 0.6% of ninhydrin.

The intermediate mixture is reacted at 100° C. for 10 minutes. After cooling down to room temperature naturally, add 150 μL of 95% alcohol, and mix evenly to get a final mixed solution. Measure absorbance of the final mixed solution at the wavelength of 570 nm (OD₅₇₀). Lastly, make a standard curve by absorbance of a set of the amino acid standard solutions obtained. Then, total concentration of amino acids in the test sample is calculated according to the standard curve.

Among the strains tested, a strain capable of decomposing fish scales is obtained and named as strain CHB200.

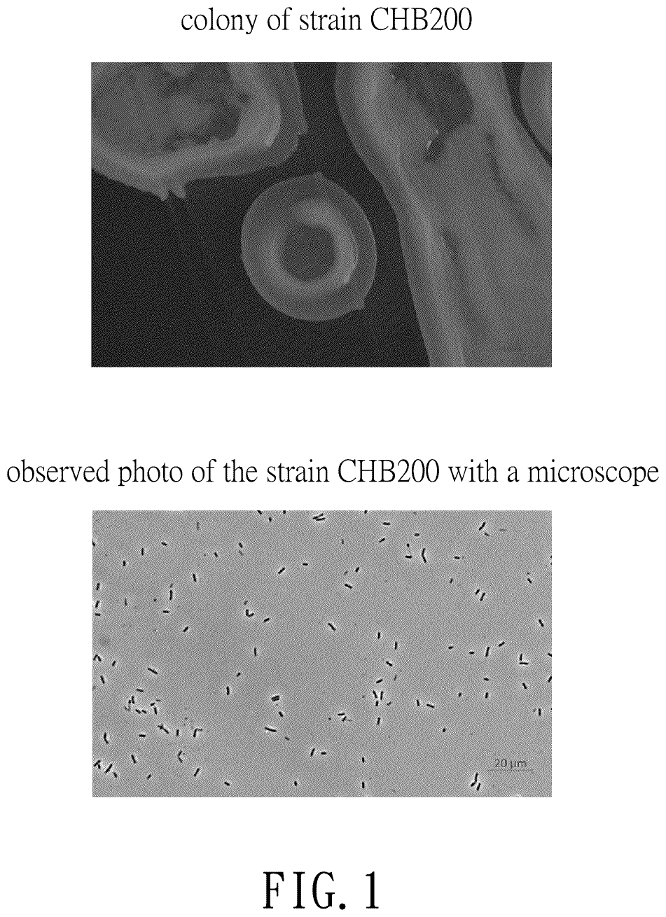

Refer to FIG. 1, a colony of the strain CHB200 is irregular round and transparent to white colored. While being observed under a microscope, the strain CHB200 is a rod with two blunt round ends. Compare 16S rRNA sequence of the strain CHB200 with sequence in Genbank database and the result shows that the strain CHB200 and the Bacillus velezensis B268 have 98% similarity in 16S rRNA sequence. 16S rRNA of the strain CHB200 is disclosed in SEQ ID NO:1 of sequence listing.

### <Test of Fish Scale Fermentation Ability of the Strain CHB200>

Inoculate the strain CHB200 into 3 ml of LB broth and culture at 37° C. with shaking at 200 rpm for 24 hours to get a bacterial solution. Then, take and inoculate the bacterial solution with OD₆₀₀ of 10 therein into 100 mL of M3 fermentation medium containing 33% fish scale powder and ferment at 37° C. to get a fermentation broth. Measure total concentration of peptides and amino acids in the fermentation broth before fermentation (0 hour) and after fermentation for 16 hrs, 24 hrs, 40 hrs, and 48 hrs. The concentration of peptides and amino acids minus the concentration of amino acids leaves the concentration of peptides.

A method of measuring total concentration of amino acids used in the test is the same as the above method using ninhydrin reagent. A method of measuring total concentration of peptides and amino acids used is using o-Phthaldialdehyde (OPA) and described briefly below.

First, prepare Leucine standard solutions. Take and add 10 μL Leucine standard solutions and test samples respectively into wells of a 96-well plate. Then add 100 μL OPA solution into the standard solutions and the samples and immediately measure absorbance of the standard solutions and the samples at the wavelength of 340 nm (OD₃₄₀). Next, make a standard curve by absorbance of the standard solutions obtained and calculate the concentration of total concentration of peptides and amino acids in the respective samples tested according to the standard curve. The result obtained by the OPA method minus the total concentration of amino acids obtained by the ninhydrin reagent method leaves total concentration of peptides in the sample tested.

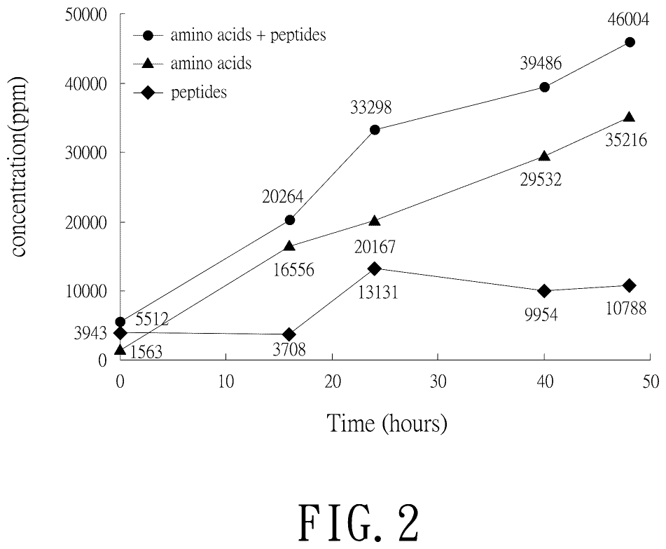

Refer to FIG. 2 and Table 2, the total concentration of amino acids and peptides in the fermentation broth is indeed increased significantly along with the increased fermentation time.

TABLE 2

| | 0 hr | 16 hrs | 24 hrs | 40 hrs | 48 hrs |
|---|---|---|---|---|---|
| total | 5512 | 20264 | 33298 | 39486 | 46004 |
| concentration | | | | | |
| of amino | | | | | |
| acids and | | | | | |
| peptides | | | | | |
| (ppm) | | | | | |
| total | 1569 | 16556 | 20167 | 29532 | 35216 |
| concentration | | | | | |
| of amino | | | | | |
| acids (ppm) | | | | | |
| total | 3943 | 3708 | 13131 | 9954 | 10788 |
| concentration | | | | | |
| of peptides | | | | | |
| (ppm) | | | | | |

### <Large Scale Fermentation of Fish Scales by the Strain CHB200>

First, activate the strain CHB200 to get a bacterial solution. Inoculate the bacterial solution in LB broth, adjust final volume into 100 mL, and incubate at 37° C. with shaking for about 16 hours to get a saturated bacterial solution. Pour the saturated bacterial solution into a 10 L fermenter and continuously incubate at 37° C. with shaking for about 2-4 hours until OD₆₀₀ of the bacterial solution reaches 4.0. Then, pour the bacterial solution with OD₆₀₀ of 4.0 into a 100-1000 L fermenter and incubate at 37° C. with shaking for about 2-4 hours until OD₆₀₀ reaches 4.0 and obtain a fermented bacterial solution.

Washed and cleaned fish scale material is placed into a 100 L-10 T fermenter and then add buffer salts and pure water to get a M3 fermentation culture medium containing fish scale materials. Then, the M3 fermentation culture medium is placed under 121° C. and 1.5 atm to be sterilized for 30 minutes. The sterilized M3 fermentation culture medium is cooled down to 30-45° C. and added with the above fermented bacterial solution to get a fermentation broth. The fermentation broth is fermented at 37° C. and stirred continuously for introduction of air. The fermentation time is 12-24 hours to obtain a primary product of fermentation. A composition of the above fermentation broth contains 7-10% (w/v) of fermented bacterial solution, 15-40% (w/v) of fish scale materials, 0.12% (w/v) of buffer salts, and the rest percentage of pure water. The preferred ratio of the fish scale materials in the fermentation broth is 20-35% (w/v) and the preferred fermentation time is 16-20 hours. The composition of the M3 fermentation culture medium is shown in Table 1.

The primary product of fermentation is centrifuged at 3000-5000 rpm or 9000-11000 rpm by a decanter centrifuge or tubular centrifuge. Keep supernatant to obtain a debris-free fermentation broth.

Then, the debris-free fermentation broth is centrifuged at 9000-12000 rpm or 9000-11000 rpm by a disc centrifuge or a tubular centrifuge. Remove bacteria sedimentation to get a clear fermentation broth and measure total concentration of amino acids, total concentration of peptides, and nitrogen content of the clear fermentation broth.

Next, the clear fermentation broth is concentrated at 45-55° C. by a vacuum low temperature evaporator for 3-6 hours to get a concentrated fermentation broth. And measure total concentration of amino acids, total concentration of peptides, and nitrogen content of the concentrated fermentation broth.

Methods of measuring the total concentration of amino acids and peptides are as shown in the above paragraphs related to the isolation and screening of Bacillus velezensis CHB200 and the test of fish scale fermentation ability of the strain CHB200 mentioned above, while a method of measuring nitrogen content is described briefly below.

Mount a homogeneous test sample into a tin foil capsule and add silicon dioxide (SiO₂) as an adsorbent. The capsule is sealed into a cylinder or a cube. Then, use an elemental analyzer (Flash 2000, Thermo Fisher Scientific) to measure nitrogen and carbon contents in the test sample. Moreover, protein content in the test sample can be estimated by multiplying the nitrogen content by a factor of 6.25 since the average nitrogen content of proteins is found to be about 16 percent.

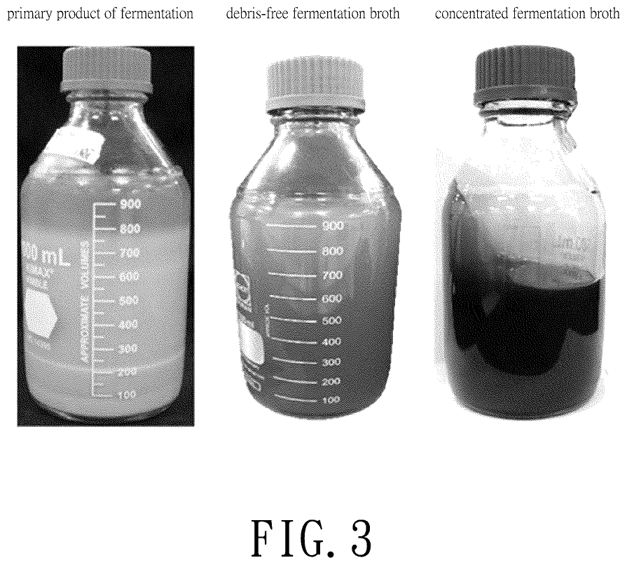

A composition analysis of the clear fermentation broth is performed by SGS Certification Taiwan and the total concentration of peptides, amino acids, nitrogen content, and protein content in the clear fermentation broth are respectively 28164 ppm, 73080 ppm, 1.7%, and 10.56%. In the concentrated fermentation broth, the total concentration of peptides, amino acids, nitrogen content, and protein content are respectively 120510 ppm, 365015 ppm, 6.9%, and 43.125%. Refer to FIG. 3, photos of the primary product of fermentation (with debris), the debris-free fermentation broth, and the concentrated fermentation broth are provided.

According to the above test results, it is learned that the strain CHB200 of the Bacillus velezensis capable of decomposing fish scales is having good fish scale decomposing ability and able to be applied to large-scale fermentation due to the effective decomposition of fish scales within a short period of time. Thus, the strain CHB200 has potential for industrial applications. Moreover, the fermentation broth obtained is rich in amino acids and peptides and thus able to be applied to agricultural fertilizer for boosting seedling growth of the Gramineae.

In a preferred embodiment, the Gramineae mentioned above includes rice, oat, wheat, barley, rye, sorghum, millet, sugarcane, maize, Job's tears, lemongrass, bamboo, water bamboo, herbage, tall fescue, bentgrass, and annual meadow grass.

A fermentation broth obtained by decomposition of fish scale material by the strain CHB200 is further used for preparation of a fish-scale amino acid peptide solution which is applied to Gramineae seedlings for growth promotion. The following are tests for showing growth promotion in the Gramineae seedlings by the fish-scale amino acid peptide solution. The concentration of the peptides and amino acids in the fish-scale amino acid peptide solution is 50-500 ppm, while 100 ppm, 250 ppm, or 500 ppm are preferred, and the optimal concentration is 100 ppm. In the following tests, corns (maize) are test objects, and the concentration of the peptides and amino acids in the fish-scale amino acid peptide solution being sprayed to the leaf surface of the corns is 100 ppm.

### Experiment Material and Method

The fermentation broth obtained by decomposition of fish scales by the strain CHB200 is diluted with water to get a fish-scale amino acid peptide solution in which a total concentration of amino acids and peptides is 100 ppm.

Corn seeds used are seeds of Glutinous Corn, White waxy corn from Known-You Seed Co., Ltd. Planting medium used is peat soil (GRAMOFLOR) and vermiculite (south sea vermiculite No. 2, fine diameter) in a ratio of 3:1. The daytime/nighttime temperature of a growth environment is set at 26/24° C. with 16 hours of daylight and 8 hours of darkness. 7 days after sowing, screen out 10 seedlings with uniform growth for both the control group and the treatment group. Then, leaf surfaces of the respective seedlings are applied with water or the fish-scale amino acid peptide solution on days 14 and 21 after sowing. The control group and the treatment group are respectively applied with water and 100 ppm fish-scale amino acid peptide solution. On day 28 after sowing, analyze physiological data such as the number of leaves, plant height, leaf width, leaf chlorophyll concentrations measured by SPAD 502 Plus (Spectrum Technologies Inc.), fresh weight, and dry weight of the corn seedlings of the control group and the treatment group. The data is analyzed by Excel Student's t-test function (*p<0.05; **p<0.01; ***p<0.001).

### Experiment Results

The corn seedlings are provided with sufficient light and water. Then, observe and take photos to record the growth of the corn seedlings sprayed with water (control group) and 100 ppm fish-scale amino acid peptide solution (treatment group) every day.

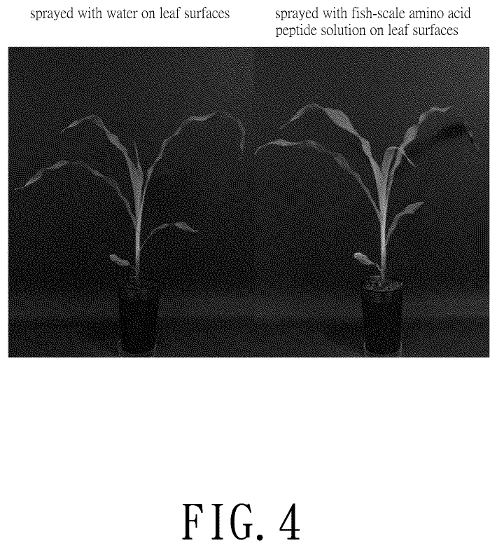

FIG. 4 is a photo showing the growth of the corn seedlings in the control group and the treatment group at day 28. Refer to the photo shown in FIG. 4, it is learned that under conditions with sufficient light and water, the corn seedling sprayed with the fish-scale amino acid peptide solution on leaf surfaces twice obviously grows thicker and taller compared with the corn seedling sprayed with water on leaf surfaces twice.

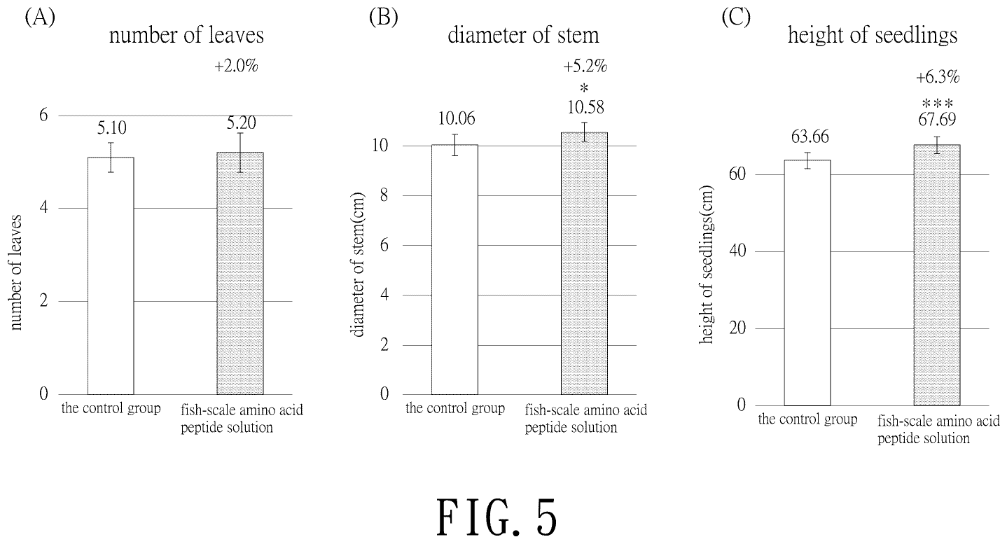

Compared with the corn seedling sprayed with water on the leaf surfaces, the corn seedling sprayed with the fish-scale amino acid peptide solution twice on the leaf surfaces has the following improvements. The number of the leaves is increased by 2% (refer to FIG. 5A). The diameter of the stem is increased by 5.2% (refer to FIG. 5B), and the height of the seedling is increased by 6.3% (refer to FIG. 5C). The number of repetitive test samples is 10 (n=10, *p<0.05; **p<0.01; ***p<0.001).

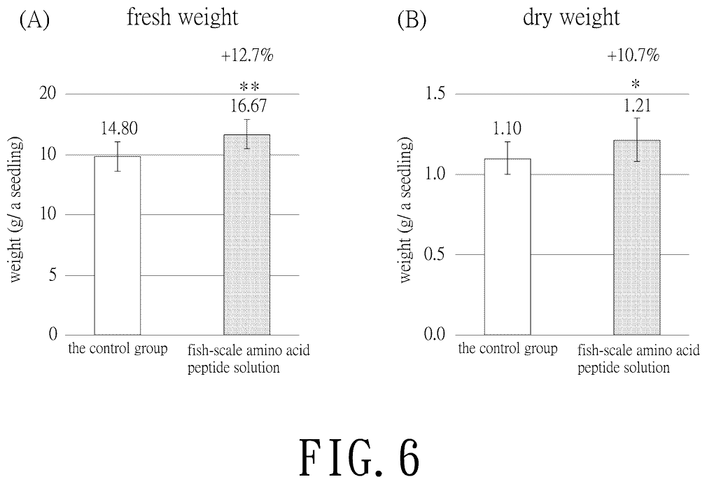

After taking photos on day 28, the above-ground part is harvested and weighted by Shimadzu AP224X analytical balance to get weight of the above-ground fresh biomass. Then, the above-ground fresh biomass is placed and dried in a large type hot air circulation oven (LENON, CO-3). Use the analytical balance to weight above-ground dry biomass. The related test data is processed and analyzed with Microsoft Office Excel software tool. Compared with the corn seedling sprayed with water on its leaf surfaces, the corn seedling sprayed with the fish-scale amino acid peptide solution twice on its leaf surfaces has a higher fresh weight and higher dry weight, which are respectively increased by 12.7% (refer to FIG. 6A) and 10.7% (refer to FIG. 6B). The number of repetitive test samples is 10 (n=10, *p<0.05; **p<0.01; ***p<0.001).

In summary, the fish-scale amino acid peptide solution obtained by decomposition of fish scales using Bacillus velezensis according to the present invention, indeed promotes the growth of corn seedlings. Compared with the control group, the number of leaves, the diameter of the stem, the length of the seedling, the fresh weight, and the dry weight of the treatment group are respectively increased by 2%, 5.2%, 6.3%, 12.7%, and 10.7%. During the tests, the amino acid peptide solutions with different concentrations applied all showed no phytotoxicity. Therefore, the fish-scale amino acid peptide solution obtained by decomposition of fish scales using Bacillus velezensis and applied to the leaf surface of the Gramineae not only makes the seedlings of the Gramineae grow healthy and strong and promotes seedling growth of the Gramineae, but also helps improve field management and yield of the Gramineae.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalent.

What is claimed is:

1. A use of an amino acid peptide solution produced by decomposition of fish scales using Bacillus velezensis for healthy and strong growth of seedlings of Gramineae is provided; the Bacillus velezensis is deposited in Leibniz Institute DSMZ-German Collection of Microorganisms and Cell Cultures GmbH with an accession number of DSM 34894; a fish scale material is decomposed by a bacterial solution containing the Bacillus velezensis to produce the fish-scale amino acid peptide solution; the fish-scale amino acid peptide solution is applied to leaf surfaces of the seedlings of the Gramineae and able to promote healthy and strong growth of the seedlings of the Gramineae specifically and effectively; the fish-scale amino acid peptide solution contains peptides and amino acids.

2. The use as claimed in claim 1, wherein a concentration of the peptides and amino acids in the fish-scale amino acid peptide solution is ranging from 50 ppm to 500 ppm.

3. The use as claimed in claim 2, wherein the concentration of the peptides and amino acids in the fish-scale amino acid peptide solution is 100 ppm, 250 ppm, or 500 ppm.

4. The use as claimed in claim 3, wherein the Gramineae includes rice, oat, wheat, barley, rye, sorghum, millet, sugarcane, maize, Job's tears, lemongrass, bamboo, water bamboo, herbage, tall fescue, bentgrass, and annual meadow grass.

5. The use as claimed in claim 4, wherein the Gramineae is maize.

6. The use as claimed in claim 2, wherein the Gramineae includes rice, oat, wheat, barley, rye, sorghum, millet, sugarcane, maize, Job's tears, lemongrass, bamboo, water bamboo, herbage, tall fescue, bentgrass, and annual meadow grass.

7. The use as claimed in claim 6, wherein the Gramineae is maize.

8. The use as claimed in claim 1, wherein the Gramineae includes rice, oat, wheat, barley, rye, sorghum, millet, sugarcane, maize, Job's tears, lemongrass, bamboo, water bamboo, herbage, tall fescue, bentgrass, and annual meadow grass.

9. The use as claimed in claim 8, wherein the Gramineae is maize.

10. A use of an amino acid peptide solution produced by decomposition of fish scales using Bacillus velezensis for growth promotion of Gramineae is provided; the Bacillus velezensis is deposited in Leibniz Institute DSMZ-German Collection of Microorganisms and Cell Cultures GmbH with an accession number of DSM 34894; a fish scale material is decomposed by a bacterial solution containing the Bacillus velezensis to produce the fish-scale amino acid peptide solution; the fish-scale amino acid peptide solution is applied to leaf surfaces of seedlings of the Gramineae and able to promote growth of the Gramineae specifically and effectively; the fish-scale amino acid peptide solution contains peptides and amino acids.

11. The use as claimed in claim 10, wherein a concentration of the peptides and amino acids in the fish-scale amino acid peptide solution is ranging from 50 ppm to 500 ppm.

12. The use as claimed in claim 11, wherein the concentration of the peptides and amino acids in the fish-scale amino acid peptide solution is 100 ppm, 250 ppm, or 500 ppm.

13. The use as claimed in claim 12, wherein the Gramineae includes rice, oat, wheat, barley, rye, sorghum, millet, sugarcane, maize, Job's tears, lemongrass, bamboo, water bamboo, herbage, tall fescue, bentgrass, and annual meadow grass.

14. The use as claimed in claim 13, wherein the Gramineae is maize.

15. The use as claimed in claim 11, wherein the Gramineae includes rice, oat, wheat, barley, rye, sorghum, millet, sugarcane, maize, Job's tears, lemongrass, bamboo, water bamboo, herbage, tall fescue, bentgrass, and annual meadow grass.

16. The use as claimed in claim 15, wherein the Gramineae is maize.

17. The use as claimed in claim 10, wherein the Gramineae includes rice, oat, wheat, barley, rye, sorghum, millet, sugarcane, maize, Job's tears, lemongrass, bamboo, water bamboo, herbage, tall fescue, bentgrass, and annual meadow grass.

18. The use as claimed in claim 17, wherein the Gramineae is maize.


SEQUENCE LISTING (1 sequence)

SEQ ID NO: 1 - RNA, 1502 nt, Bacillus velezensis
  source 1..1502: mol_type = rRNA
gacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgggacagatgattggggtgaagtcgtaacaaggtagccgtatcggaagg